(19) United States
(12) Patent Application Publication
(10) Pub. No.: US 2013/0029348 A1
(43) Pub. Date: Jan. 31, 2013

(54) METHODS OF ELONGATING NUCLEIC ACIDS

(71) Applicants: Ryan Nathan Ptashkin, Bethesda, MD (US); Wenlong Jaing, 8429 Castle Pines Dr, WI (US)

(72) Inventors: Ryan Nathan Ptashkin, Bethesda, MD (US); Wenlong Jaing, 8429 Castle Pines Dr, WI (US)

(73) Assignee: OPGEN, INC., Gaithersburg, MD (US)

(21) Appl. No.: 13/190,964

(22) Filed: Jul. 26, 2011

(51) Int. Cl. C12Q 1/68

(52) U.S. Cl. 435/6.15

(57) ABSTRACT

The invention generally relates to methods for elongating nucleic acid, such as DNA, on a charged substrate. In certain embodiments, methods of the invention involve contacting an applicator to a sample including a nucleic acid, and swabbing the applicator on a charged substrate, thereby elongating the nucleic acid on the substrate.

### FIELD OF THE INVENTION

The invention generally relates to methods for elongating nucleic acids, such as DNA, on a charged substrate.

### BACKGROUND

Physical genomic mapping using restriction endonucleases can provide accurate information about the nucleic acid sequences of various organisms. Optical mapping can be used to produce ordered restriction maps that are visualized using fluorescence microscopy.

In optical mapping, nucleic acids are digested by restriction enzymes on a glass surface. The nucleic acids are fixed and elongated on the surface to provide access to restriction sites for the enzymes. Polydimethylsiloxane (PDMS) coated microchannels are good surfaces for fixing stretched single molecules for optical mapping purposes. In this process, a microchannel is temporarily sealed to a charged glass substrate by mechanical placement, a small volume of nucleic acid solution is flowed into the resulting microchannels, excess nucleic acid solution is removed from the surface/channel interface, followed by the removal of the microchannel to continue processing for optical mapping protocols.

A problem with deposition techniques that use microchannels is that the manual intervention required significantly hinders development of high throughput optical mapping protocols. Further, standard microchannel protocols are not optimal with respect to scalability and automation for high-throughput optical mapping.

There is a need for methods that provide a mechanism of delivering stretched individual nucleic acid molecules to a substrate for high throughput optical mapping.

### SUMMARY

The invention generally relates to methods for elongating nucleic acids, such as DNA, on a charged substrate. Methods of the invention use an applicator, such as rod, a cotton-tipped rod, or a sterile loop, to deposit nucleic acids onto a charged substrate. One end of the applicator is applied to a solution containing nucleic acid to be analyzed. That same end of the applicator is then dragged or swabbed on at least as portion of the charged substrate. The swabbing action on the charged substrate deposits individually stretched or elongated DNA molecules on the charged substrate. This process may be accomplished by hand or may be automated. The invention eliminates the need for bulk flow based nucleic acid deposition techniques and also eliminates the need for microchannels. Methods of the invention allow for automation of optical mapping processes and provide increased sensitivity.

In certain embodiments, the applicator is used to swab the entire surface of the charged substrate. Methods of the invention may also involve obtaining a purified nucleic acid solution prior to contacting the applicator the sample. Any applicator may be used that will retain nucleic acid and that will allow depositing of the nucleic acid on a charged substrate. Exemplary applicators include a rod, a cotton-tipped rod, or a sterile loop. The substrate may be composed of any material that is compatible with optical mapping. In certain embodiments, a glass substrate is used. The glass substrate may be coated with silane.

The applicator may also be modified for use in sample preparation. This may include, but not be limited to, the addition of a magnet, magnetic beads, syringe pump, and/or plunger, to be used with nucleic acid extraction buffers. In these embodiments, methods of the invention allow taking a sample from appropriate growth media or directly from sample, performing DNA extraction, and depositing the DNA on a charged glass substrate.

Another aspect of the invention provides a method for generating a physical map of a genome of an organism. These methods of the invention involve contacting an applicator to sample comprising nucleic acids, swabbing the applicator on a charged substrate such that the nucleic acids are elongated and fixed on the substrate so that the nucleic acids remain accessible for enzymatic reactions, digesting the nucleic acids enzymatically to produce one or more restriction digests, imaging the restriction digests, and constructing an optical map from the restriction digests. Prior to the contacting step, the invention may further include obtaining a purified solution of nucleic acids.

The detected organism can be a microorganism, a bacterium, a protist, a virus, a fungus, or disease-causing organisms including microorganisms such as protozoa and multicellular parasites. In certain embodiments, the bacterium is an E. coli or an S. aureus. In particular embodiments, methods of the invention can distinguish between a community-acquired methicillin-resistant strain of S. aureus and a hospital-acquired methicillin-resistant strain of S. aureus.

The nucleic acid can be deoxyribonucleic acid (DNA), a ribonucleic acid (RNA) or can be a cDNA copy of an RNA obtained from a sample. The nucleic acid sample includes any tissue or body fluid sample, environmental sample (e.g., water, air, dirt, rock, etc.), and all samples prepared therefrom. In certain embodiments, the nucleic acid includes substantially all genomic DNA of the bacterium. In other embodiments, the nucleic acid includes a transcriptome of the bacterium. Furthermore, the nucleic acid may be labeled prior to or upon application to the substrate; or otherwise modified to increase the ease of detection.

### BRIEF DESCRIPTION OF THE DRAWINGS

FIG. 1 is a photograph showing an image of stretched E. coli K12 DNA using methods of the invention.

### DETAILED DESCRIPTION

Standard optical mapping techniques involve elongating nucleic acids in PDMS microchannels treated with oxygen plasma to render the channels hydrophilic. Hydrophilicity combined with channel geometry results in viscous drag forces produced by the bulk flow of solution which, in turn, is capable of exerting sufficient stretching force on nucleic acids to elongate the nucleic acids on a charged surface. In contrast, methods of the invention involve contacting an applicator to a sample including nucleic acids, and swabbing the applicator on a charged substrate, thereby elongating the nucleic acids on the substrate. Methods of the invention eliminate the need for oxygen plasma treatment of PDMS microchannels, and also eliminate the need for addition of surfactant to the nucleic acid solution to account for loss in hydrophilicity. Further, methods of the invention operate without the need for bulk flow of solutions.

In certain embodiments, methods of the invention may also involve obtaining a purified solution of the nucleic acids prior to contacting the applicator the sample. Methods of extracting nucleic acids and methods of purifying biological samples are known in the art. See for example, Maniatis et al., Molecular Cloning: A Laboratory Manual, Cold Spring Harbor, N.Y. (1982).

Methods of the invention may be used with any applicator that will retain nucleic acid after it has be contacted to the sample, and that will subsequently deposit the nucleic acid once the applicator comes in contact with the charged substrate. Exemplary applicators include a rod (such as a glass, polymer, or metal rod), a rod having a tip made of an absorbent material (such as cotton), or a sterile loop.

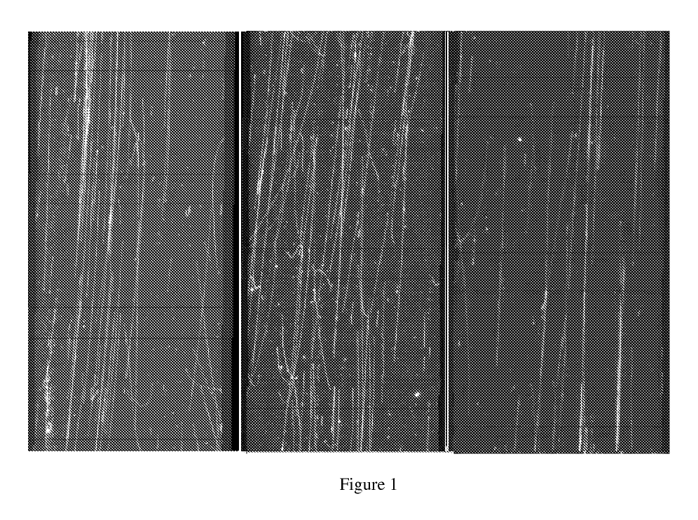

Once the applicator has been contacted to the solution containing the nucleic acid, the applicator is swabbed or dragged on the charged surface. Without being limited by any particular theory or mechanism of action, it is believed that the stretching mechanism used with this invention most likely employs the force of a receding meniscus along the length of the swab area, with an origin at one or multiple locations. FIG. 1 shows an image of stretched E. coli K12 DNA using methods of the invention. FIG. 1 shows that the stretch high molecular weight DNA is in a directional pattern, which is important for optical mapping.

The surface can be composed of any material that is suitable for optical mapping and is compatible with nucleic acids. Exemplary materials include polymers, ceramics, glass, or metals. In a preferred embodiment, the surface is glass, such as a microscope slide. Because a net positive charge is require to capture/retain nucleic acids, the surface includes silanes to impart a net positive charge to the surface.

The applicator may also be modified or used in such a way as to prepare a nucleic acid sample. This may include, but not be limited to, the addition of a magnet, magnetic beads, syringe pump, and/or plunger, to be used with nucleic acid extraction buffers. In these embodiments, methods of the invention allow taking a sample from appropriate growth media or directly from sample, performing DNA extraction, and depositing the DNA on a charged glass substrate.

Another aspect of the invention provides a method for generating a physical map of a genome of an organism. These methods of the invention involve contacting an applicator to sample comprising nucleic acid, swabbing the applicator on a charged substrate such that the nucleic acid is elongated and fixed on the substrate so that the nucleic acid remains accessible for enzymatic reactions, digesting the nucleic acid enzymatically to produce one or more restriction digests, imaging the restriction digests, and constructing an optical map from the restriction digests.

The sample may be a human tissue or body fluid. A tissue is a mass of connected cells and/or extracellular matrix material, e.g. skin tissue, nasal passage tissue, CNS tissue, neural tissue, eye tissue, liver tissue, kidney tissue, placental tissue, mammary gland tissue, placental tissue, gastrointestinal tissue, musculoskeletal tissue, genitourinary tissue, bone marrow, and the like, derived from, for example, a human or other mammal and includes the connecting material and the liquid material in association with the cells and/or tissues.

A body fluid is a liquid material derived from, for example, a human or other mammal. Such body fluids include, but are not limited to, mucous, blood, plasma, serum, serum derivatives, bile, blood, maternal blood, phlegm, saliva, sweat, amniotic fluid, mammary fluid, urine, and cerebrospinal fluid (CSF), such as lumbar or ventricular CSF. A sample may also be a fine needle aspirate or biopsied tissue. A sample also may be media containing cells or biological material.

The sample may also be an environmental sample such as water, air, dirt, rock, etc. In other embodiments, the sample is a food sample.

In certain embodiments, methods of the invention may also involve obtaining a purified solution of the nucleic acids prior to introducing the non-ionic surfactant. Methods of extracting nucleic acids and methods of purifying biological samples are known in the art. See for example, Maniatis et al., Molecular Cloning: A Laboratory Manual, Cold Spring Harbor, N.Y. (1982).

Optical mapping is a single-molecule technique for production of ordered restriction maps from a single DNA molecule (Samad et al., Genome Res. 5:1-4, 1995). During some applications, individual fluorescently labeled DNA molecules are elongated and fixed on the surface using methods of the invention. The added endonuclease cuts the DNA at specific points, and the fragments are imaged. Id. Exemplary endonucleases include BglII, NcoI, XbaI, and BamHI. Exemplary combinations of restriction enzymes include:

| | | |
|---|---|---|
| AflII | ApaLI | BglII |
| AflII | BglII | NcoI |
| ApaLI | BglII | NdeI |
| AflII | BglII | MluI |
| AflII | BglII | PacI |
| AflII | MluI | NdeI |
| BglII | NcoI | NdeI |
| AflII | ApaLI | MluI |
| ApaLI | BglII | NcoI |
| AflII | ApaLI | BamHI |
| BglII | EcoRI | NcoI |
| BglII | NdeI | PacI |
| BglII | Bsu36I | NcoI |
| ApaLI | BglII | XbaI |
| ApaLI | MluI | NdeI |
| ApaLI | BamHI | NdeI |
| BglII | NcoI | XbaI |
| BglII | MluI | NcoI |
| BglII | NcoI | PacI |
| MluI | NcoI | NdeI |
| BamHI | NcoI | NdeI |
| BglII | PacI | XbaI |
| MluI | NdeI | PacI |
| Bsu36I | MluI | NcoI |
| ApaLI | BglII | NheI |
| BamHI | NdeI | PacI |
| BamHI | Bsu36I | NcoI |
| BglII | NcoI | PvuII |
| BglII | NcoI | NheI |
| BglII | NheI | PacI |

Restriction maps can be constructed based on the number of fragments resulting from the digest. Id. Generally, the final map is an average of fragment sizes derived from similar molecules. Id.

Optical mapping and related methods are described in U.S. Pat. No. 5,405,519, U.S. Pat. No. 5,599,664, U.S. Pat. No. 6,150,089, U.S. Pat. No. 6,147,198, U.S. Pat. No. 5,720,928, U.S. Pat. No. 6,174,671, U.S. Pat. No. 6,294,136, U.S. Pat. No. 6,340,567, U.S. Pat. No. 6,448,012, U.S. Pat. No. 6,509,158, U.S. Pat. No. 6,610,256, and U.S. Pat. No. 6,713,263. All the cited patents are incorporated by reference herein in their entireties.

Optical Maps are constructed as described in Reslewic et al., Appl Environ Microbiol. 2005 September; 71 (9):5511-22, incorporated by reference herein. Briefly, individual chromosomal fragments from test organisms are immobilized on derivatized glass by virtue of electrostatic interactions between the negatively-charged DNA and the positively-charged surface, digested with one or more restriction endonuclease, stained with an intercalating dye such as YOYO-1 (Invitrogen) and positioned onto an automated fluorescent microscope for image analysis. Since the chromosomal fragments are immobilized, the restriction fragments produced by digestion with the restriction endonuclease remain attached to the glass and can be visualized by fluorescence microscopy, after staining with the intercalating dye. The size of each restriction fragment in a chromosomal DNA molecule is measured using image analysis software and identical restriction fragment patterns in different molecules are used to assemble ordered restriction maps covering the entire chromosome.

Restriction mapping, e.g., optical mapping, can be used in a variety of applications. For example, the methods featured herein can be used to determine a property, e.g., physical and/or chemical property, e.g., size, length, restriction map, weight, mass, sequence, conformational or structural change, pKa change, distribution, viscosity, rates of relaxation of a labeled and/or non-labeled molecule, e.g., an amplicon (e.g., PCR product), of a portion of a genome (e.g., a chromosome), or of an entire genome.

Optical mapping can also be used to identify various organisms, e.g., viruses and prions, and various microorganisms, e.g., bacteria, protists, and fungi, whose genetic information is stored as DNA or RNA by correlating the restriction map of a nucleic acid of an organism with a restriction map database. Such identification methods can be used in diagnosing a disease or disorder. Methods of identifying organisms by restriction mapping are described, e.g., in a U.S. patent application Ser. No. 12/120,586, filed on May 14, 2008, incorporated herein by reference. The methods featured herein can also be used in other diagnostic applications, for example, imaging specific loci or genetic regions for individuals or populations to help identify specific diseases or disorders. Other uses of the methods will be apparent to those skilled in the art.

The methods described herein can be used in a variety of settings, e.g., to identify an organism in a human or a non-human subject, in food, in environmental sources (e.g., food, water, air), and in industrial settings. The featured methods also include methods of diagnosing a disease or disorder in a subject, e.g., a human or a non-human subject, and treating the subject based on the diagnosis. The method includes: obtaining a sample comprising an organism from the subject; imaging a nucleic acid from the organism; obtaining a restriction map of said nucleic acid; identifying the organism by correlating the restriction map of said nucleic acid with a restriction map database; and correlating the identity of the organism with the disease or disorder.

As discussed above, various organisms can be identified by the methods discussed herein and therefore various diseases and disorders can be diagnosed by the present methods. The organism can be, e.g., a cause, a contributor, and/or a symptom of the disease or disorder. In one embodiment, more than one organism can be identified by the methods described herein, and a combination of the organisms present can lead to diagnosis. Skilled practitioners would be able to correlate the identity of an organism with a disease or disorder. For example, the following is a non-exhaustive list of some diseases and bacteria known to cause them: tetanus—Clostridium tetani; tuberculosis—Mycobacterium tuberculosis; meningitis—Neisseria meningitidis; botulism—Clostridium botulinum; bacterial dysentry—Shigella dysenteriae; lyme disease—Borrelia burgdorferi; gasteroenteritis—E. coli and/or Campylobacter spp.; food poisoning—Clostridium perfringens, Bacillus cereus, Salmonella enteriditis, and/or Staphylococcus aureus. These and other diseases and disorders can be diagnosed by the methods described herein.

Once a disease or disorder is diagnosed, a decision about treating the subject can be made, e.g., by a medical provider or a veterinarian. Treating the subject can involve administering a drug or a combination of drugs to ameliorate the disease or disorder to which the identified organism is contributing or of which the identified organism is a cause. Amelioration of the disease or disorder can include reduction in the symptoms of the disease or disorder. The drug administered to the subject can include any chemical substance that affects the processes of the mind or body, e.g., an antibody and/or a small molecule, The drug can be administered in the form of a composition, e.g., a composition comprising the drug and a pharmaceutically acceptable carrier. The composition can be in a form suitable for, e.g., intravenous, oral, topical, intramuscular, intradermal, subcutaneous, and anal administration. Suitable pharmaceutical carriers include, e.g., sterile saline, physiological buffer solutions and the like. The pharmaceutical compositions may be additionally formulated to control the release of the active ingredients or prolong their presence in the patient's system. Numerous suitable drug delivery systems are known for this purpose and include, e.g., hydrogels, hydroxymethylcellulose, microcapsules, liposomes, microemulsions, microspheres, and the like. Treating the subject can also include chemotherapy and radiation therapy.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

1. A method for elongating a nucleic acid on a substrate, the method comprising:
contacting a sample comprising a nucleic acid with an applicator that comprises a material that temporarily retains nucleic acid, thereby causing the nucleic acid to be temporarily retained on the applicator;
contacting the applicator comprising the retained nucleic acid to a charged substrate; and
moving the applicator along at least a portion of the charged substrate while the applicator is in contact with the substrate, thereby causing the retained nucleic acid to be deposited and elongated on the substrate.

2. The method according to claim 1, wherein the applicator comprises a tip.

3. The method according to claim 2, wherein the tip comprises a cotton material.

4. The method according to claim 2, wherein the tip is a sterile loop.

5. The method according to claim 1, wherein the substrate is glass.

6. The method according to claim 5, wherein the glass is coated with silanes.

7. The method according to claim 1, the applicator comprises a tip that is modified to assist in sample preparation.

8. The method according to claim 7, wherein the modification is selected from the group consisting of: a magnet, magnetic beads present in the tip; nucleic acid extraction buffer coating the tip; and a syringe pump coupled to the tip.

9. The method according to claim 1, wherein prior to the contacting step, the method further comprises obtaining a purified solution of the nucleic acid.

10. The method according to claim 1, wherein the nucleic acid is DNA.

11. A method for generating a physical map of a genome of an organism, the method comprising:
contacting a sample comprising a nucleic acid with an applicator that comprises a material that temporarily retains nucleic acid, thereby causing the nucleic acid to be temporarily retained on the applicator;
contacting the applicator comprising the retained nucleic acid to a charged substrate; and
moving the applicator along at least a portion of the charged substrate while the applicator is in contact with the substrate, such that the retained nucleic acid is elongated and fixed on the substrate so that the nucleic acid remains accessible for enzymatic reactions;
imaging the restriction digests; and
constructing an optical map from the restriction digests.

12. The method according to claim 11, wherein prior to the contacting step, the method further comprises obtaining a purified solution of the nucleic acid.

13. The method according to claim 11, wherein the nucleic acid is DNA.

14. The method according to claim 11, wherein the nucleic acid is from a microorganism.

15. The method according to claim 14, wherein the microorganism is a bacterium.

16. The method according to claim 15, wherein the bacterium is at least one species selected from the group consisting of Escherichia coli (E. coli) and Staphylococcus aureus (S. aureus).

17. The method according to claim 16, wherein the S. aureus is a community-acquired methicillin-resistant strain of S. aureus.

18. The method according to claim 16, wherein the S. aureus is a hospital-acquired methicillin-resistant strain of S. aureus.

19. The method according to claim 15, wherein the nucleic acid comprises substantially all genomic DNA of the bacterium.

20. The method according to claim 15, wherein the nucleic acid comprises a transcriptome of the bacterium.
